(19) United States
(12) Patent Application Publication
(10) Pub. No.: US 2012/0107240 A1
(43) Pub. Date: May 3, 2012

(54) Probes and methods of melanoma imaging

(75) Inventors: Shuanglong Liu, Mountain View, CA (US); Hongguang Liu, Palo Alto, CA (US); Zhen Cheng, Mountain View, CA (US)

(73) Assignee: The Board of Trustees of the Leland Stanford Junior University, Palo Alto, CA (US)

(21) Appl. No.: 13/373,072

(22) Filed: Nov. 3, 2011

Related U.S. Application Data
(60) Provisional application No. 61/409,680, filed on Nov. 3, 2010.

(51) Int. Cl. A61K 51/00; C07D 211/78; C12Q 1/02

(52) U.S. Cl. 424/1.89; 435/29; 546/328

(57) ABSTRACT

Embodiments of the present disclosure provide for labeled probes such as a 3-¹⁸F-fluoropicolinamide probe, methods of making labeled probes, pharmaceutical compositions including labeled probes, methods of using labeled probes, methods of diagnosing, localizing, monitoring, and/or assessing melanin related diseases, cancers, tumors, precancerous cells, and related biological events (e.g., malignant melanoma), using labeled probes, kits for diagnosing, localizing, monitoring, and/or assessing melanin related diseases, cancers, tumors, precancerous cells, and related biological events, using labeled probes, and the like.

### CROSS-REFERENCE TO RELATED APPLICATION

This application claims priority to U.S. provisional application entitled “PROBES AND METHODS OF MELANOMA IMAGING,” having Ser. No. 61/409,680, filed on Nov. 3, 2010, which is entirely incorporated herein by reference.

### BACKGROUND

Melanoma is a type of skin cancer. A melanoma tumor develops from abnormal melanocytes in the lower epidermis. A melanocyte is a pigment cell that can become abnormal to varying degrees, where in some cases the abnormality of a melanocyte cell is to become a melanoma cell. A melanoma cell is a malignancy (a cancer or tumor cell) that can kill surrounding tissue and can metastasize to distant sites in the body via the blood and lymph systems. The incidence and mortality rate of malignant melanoma continue to rise dramatically throughout the world and is one of the most feared neoplasms because of the high mortality associated with metastasis. Thus, there is a need to detect and identify melanoma as early as possible.

### SUMMARY

Embodiments of the present disclosure provide for labeled probes such as a 3-¹⁸F-fluoropicolinamide probe, methods of making labeled probes, pharmaceutical compositions including labeled probes, methods of using labeled probes, methods of diagnosing, localizing, monitoring, and/or assessing melanin related diseases, cancers, tumors, precancerous cells, and related biological events (e.g., malignant melanoma), using labeled probes, kits for diagnosing, localizing, monitoring, and/or assessing melanin related diseases, cancers, tumors, precancerous cells, and related biological events, using labeled probes, and the like.

An embodiment of the method of diagnosing the presence of a melanin related disease in a subject, among others, includes: administering to the subject a labeled probe as shown in FIG. 1A and described herein; imaging at least a portion of the subject; and detecting the labeled probe, wherein the location of the labeled probe corresponds to a melanin related disease.

An embodiment of diagnosing the presence of a melanin related disease in a sample, among others, includes: contacting the sample with a labeled probe as shown in FIG. 1A and discussed herein; imaging at least a portion of the sample; and detecting the labeled probe, wherein the location of the labeled probe corresponds to a melanin related disease.

An embodiment of monitoring the progress of a melanin related disease in a subject, among others, includes: administering to the subject a labeled probe as shown in FIG. 1A and discussed herein; imaging at least a portion of the subject; and detecting the labeled probe, wherein the location of the labeled probe corresponds to a melanin related disease, wherein the dimensions of the location is monitored over time.

An embodiment of monitoring the progress of a melanin related disease in a sample, among others, includes: contacting the sample with a labeled probe as shown in FIG. 1A and discussed herein; imaging at least a portion of the sample; and detecting the labeled probe, wherein the location of the labeled probe corresponds to a melanin related disease, wherein the size of the location is monitored over time.

An embodiment of monitoring the progress of a melanin related disease in a sample, among others, includes: contacting the sample with a labeled probe as shown in FIG. 1A and discussed herein; imaging at least a portion of the sample; and detecting the labeled probe, wherein the location of the labeled probe corresponds to a melanin related disease, wherein the size of the location is monitored over time.

An embodiment of the method of screening for an agent for treating a melanin related disease in a sample, among others, includes: contacting the sample with a labeled probe as shown in FIG. 1A and discussed herein, wherein a melanin related disease is present in the sample; contacting an agent with the sample; imaging at least a portion of the sample; and detecting the labeled probe, wherein the location of the labeled probe corresponds to a melanin related disease, wherein the size of the location is monitored over time.

An embodiment of probe, among others, includes: a labeled probe such as that shown in FIG. 1A and discussed herein.

An embodiment of the composition, among others, includes: a labeled probe such as that shown in FIG. 1A and discussed herein.

An embodiment of the pharmaceutical composition, among others, includes: a labeled probe such as that shown in FIG. 1A and discussed herein.

Other compositions, probes, methods, features, and advantages of the present disclosure will be, or become, apparent to one with skill in the art upon examination of the following drawings and detailed description. It is intended that all such additional structures, methods, features, and advantages be included within this description, be within the scope of the present disclosure, and be protected by the accompanying claims.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIG. 1A illustrates a labeled probe of the present disclosure. FIG. 1B illustrates embodiments of the labeled probe and a method of making a specific labeled probe.

FIG. 2 illustrates that 3-¹⁸F-fluoropicolinamide (¹⁸F-P3BZA) surprisingly has higher tumor uptake than other nicotinamide analogs tested in B16F10 xenograph murine model.

FIG. 3 illustrates the microPET quantification analysis of tumors and other organs.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, synthetic organic chemistry, biochemistry, biology, molecular biology, molecular imaging, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

By “administration” or “administering” is meant introducing a probe or a labeled probe (also referred to as the “imaging agent”) of the present disclosure into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used.

In accordance with the present disclosure, “a detectably effective amount” of the probe of the present disclosure is defined as an amount sufficient to yield an acceptable image using equipment that is available for clinical use. A detectably effective amount of the probe of the present disclosure may be administered in more than one injection. The detectably effective amount of the probe of the present disclosure can vary according to factors such as the degree of susceptibility of the individual, the age, sex, and weight of the individual, idiosyncratic responses of the individual, and the like. Detectably effective amounts of the probe of the present disclosure can also vary according to instrument and film-related factors. Optimization of such factors is well within the level of skill in the art.

As used herein, the term “host” or “subject” includes humans and mammals (e.g., cats, dogs, horses, etc.). Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications. In some embodiments, a system includes a sample and a host. The term “living subject” refers to a subject noted above that is alive and is not dead. The term “living subject” refers to the entire subject and not just a part excised (e.g., a liver or other organ) from the living subject.

The term “sample” can refer to a tissue sample, cell sample, a fluid sample, and the like. The sample may be taken from a subject. The tissue sample can include hair (including roots), buccal swabs, blood, saliva, semen, muscle, or from any internal organs. The fluid may be, but is not limited to, urine, blood, ascites, pleural fluid, spinal fluid, and the like. The body tissue can include, but is not limited to, skin, muscle, endometrial, uterine, and cervical tissue. In the present disclosure, the source of the sample is not critical.

The term “detectable” refers to the ability to detect a signal over the background signal.

The term “detectable signal” is a signal derived from non-invasive imaging techniques such as, but not limited to, positron emission tomography (PET) or single photon emission computed tomography (SPECT). The detectable signal is detectable and distinguishable from other background signals that may be generated from the subject. In other words, there is a measurable and statistically significant difference (e.g., a statistically significant difference is enough of a difference to distinguish among the detectable signal and the background, such as about 0.1%, 1%, 3%, 5%, 10%, 15%, 20%, 25%, 30%, or 40% or more difference between the detectable signal and the background) between the detectable signal and the background. Standards and/or calibration curves can be used to determine the relative intensity of the detectable signal and/or the background.

“Cancer”, as used herein, shall be given its ordinary meaning, as a general term for diseases in which abnormal cells divide without control. In particular, cancer refers to melanin related cancer. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult.

In an embodiment, cancer refers to malignant melanoma.

The phrase “melanin related diseases” can refer to malignant melanoma, hyperpigmentation, and other diseases that accompanied with change of melanin content.

### General Discussion

Embodiments of the present disclosure provide for labeled probes such as a 3-¹⁸F-fluoropicolinamide probe, methods of making labeled probes, pharmaceutical compositions including labeled probes, methods of using labeled probes, methods of diagnosing, localizing, monitoring, and/or assessing melanin related diseases, cancers, tumors, precancerous cells, and related biological events (e.g., malignant melanoma), using labeled probes, kits for diagnosing, localizing, monitoring, and/or assessing melanin related diseases, cancers, tumors, precancerous cells, and related biological events, using labeled probes, and the like. In particular, the present disclosure includes methods relating to non-invasive imaging (e.g., using positron emission tomography (PET) imaging system) using labeled probes, such as a 3-¹⁸F-fluoropicolinamide probe, in vivo.

Portions of the present disclosure discuss labeled probes while other portions describe a specific embodiment of the labeled probes, the 3-¹⁸F-fluoropicolinamide probe. Discussions focusing on the 3-¹⁸F-fluoropicolinamide probe are not limiting to the scope of the disclosure, rather those discussions are merely describing an exemplary embodiment of the present disclosure.

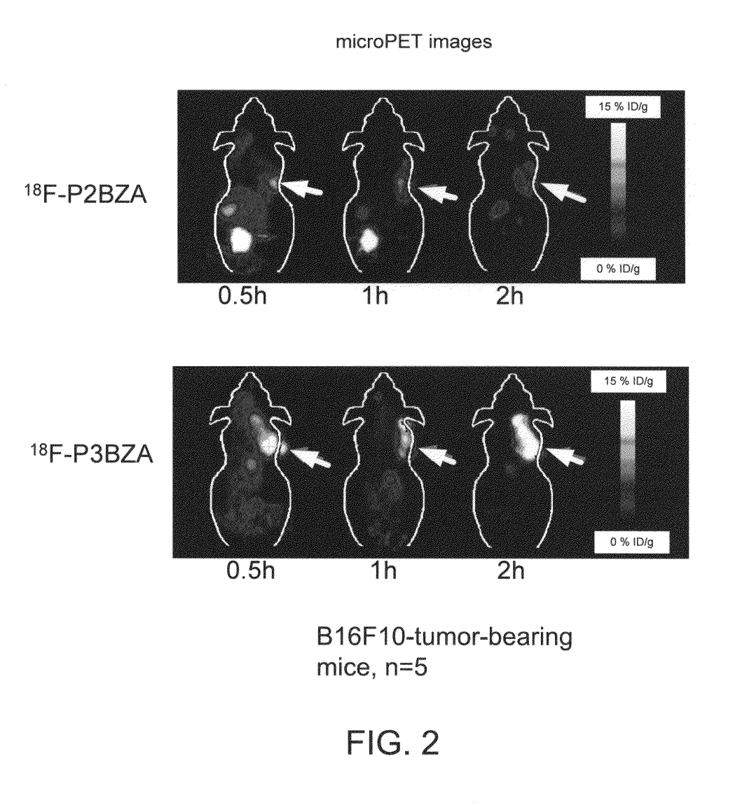

Embodiments of the present disclosure are advantageous over the other isomer of pyridinecarboxamide, nicotinamide. As discussed in the Example, the 3-¹⁸F-fluoropicolinamide (¹⁸F-P3BZA) probe surprisingly has high (e.g., 11.8±2.2, 13.2±2.4, and 14.6±3.2% ID/g at 0.5 h, 1 h, and 2 h post injection, respectively) tumor uptake than other nicotinamide analogs tested in B16F10 xenograph murine model (FIG. 2). In addition, the ¹⁸F-P3BZA probe surprisingly has significantly high (e.g., tumor to muscle ratio equals 25.3 at 2 h) contrast than that of ¹⁸F-P2BZA probe under the same scale bar, as determined by the Student test (P<0.05). The in vivo data were also achieved using the 4-¹⁸F-fluoropicolinamide (¹⁸F-P4BZA) probe for comparison performed in the same tumor model (data not shown). The tumor uptake of ¹⁸F-P4BZA probe is only slightly lower (e.g., 10.7±1.3, 10.5±1.8, and 8.7±2.3% ID/g at 0.5 h, 1 h, and 2 h post injection, respectively) than that of ¹⁸F-P3BZA probe. However, the defluorination of ¹⁸F-P4BZA probe in the murine model was very high, as evidenced by that fact of the high bone uptake of ¹⁸F-P4BZA probe (e.g., 11.3±2.9, 10.3±3.6% ID/g at 1 h, and 2 h postinjection, respectively). The high bone uptake lowered the tumor to background contrast. So, ¹⁸F-P3BZA probe showed multiple advantages and promises smooth clinical translation in terms of accurate staging, and early diagnosis of primary and metastatic malignant melanoma. Additional details are described in the Examples.

Embodiments of the present disclosure include methods for imaging a sample (e.g., tissue or cell(s)) or a subject, that includes contacting a sample with or administering to a subject a labeled probe (a ¹⁸F-P3BZA probe) and imaging with a PET imaging system. The imaging can be performed in vivo and/or in vitro. In particular, embodiments of the present disclosure can be used to image melanin related diseases, such as malignant melanoma, or related biological events. In this regard, the sample or subject, can be tested to determine if the sample or subject includes a melanin related disease or related biological events, monitor the progression (or regression) of the disease, or assess the response of the disease to treatment, image, and the like. In an embodiment, the tissue or cells can be within a subject or have been removed from a subject.

In an embodiment, the 3-¹⁸F-fluoropicolinamide probe can be imaged using imaging systems such as positron emission tomography (PET) imaging systems, single photon emission computed tomography (SPECT), and the like. In an embodiment, PET imaging is a preferred embodiment. Other types of labeled probes can use appropriate imaging systems.

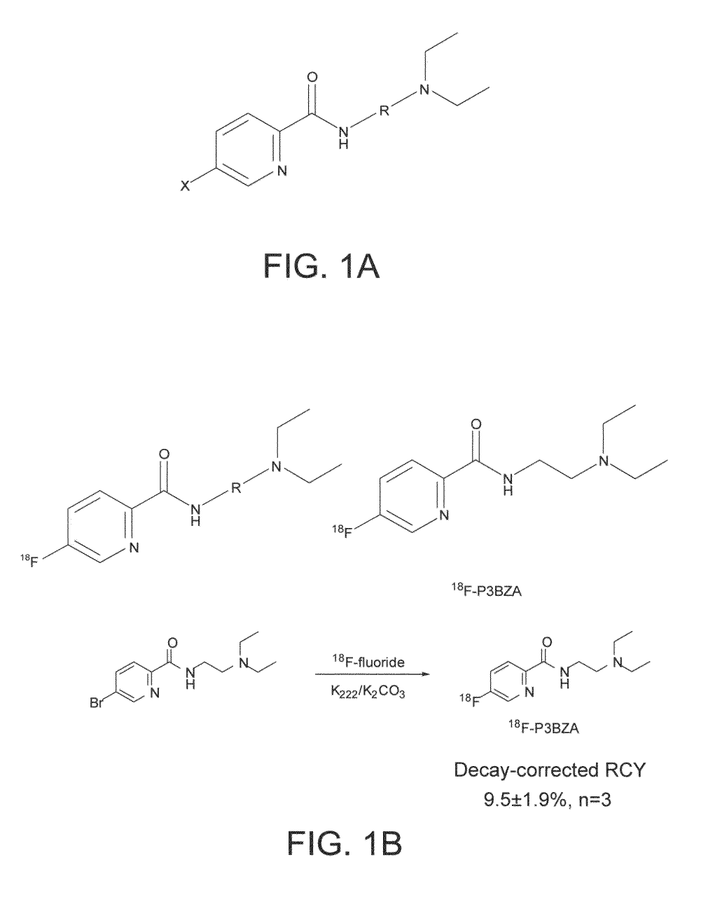

FIG. 1A illustrates a labeled probe that can be used in diagnosing, localizing, monitoring, and/or assessing melanin related diseases, cancers, tumors, precancerous cells, and related biological events (e.g., malignant melanoma). In particular, the present disclosure includes methods relating to non-invasive imaging (e.g., using positron emission tomography (PET) imaging system) using the labeled probe in vivo. In an exemplary embodiment X can be a radiolabel such as ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, or ¹⁵³Sm. In an embodiment, the radiolabel, X, can be ¹⁸F, ⁷⁶Br, or ¹²³I, ¹²⁴I or ¹³¹I, which are suitable for use in peripheral medical facilities and PET clinics. In other embodiments, the radiolabel or PET isotope, X, can include, but is not limited to, ⁶⁴Cl, ¹²⁴I, ^{76/77}Br, ⁸⁶Y, ⁸⁹Zr, or ⁶⁸Ga. In an exemplary embodiment R can be an alkyl group such as CₙH₂ₙ, where n can be 1 to 12, that can be substituted or unsubstituted and/or linear or branched, or an alkene group (e.g., 1 to 12 carbons with one or more double bonds that can be substituted or unsubstituted and/or linear or branched).

FIG. 1B illustrates embodiments of the labeled probe and a method of making a specific labeled probe. R can be a group as defined in reference to FIG. 1A. Specifically, FIG. 1B illustrates an embodiment of the 3-¹⁸F-fluoropicolinamide probe and a method of making the 3-¹⁸F-fluoropicolinamide probe.

In an embodiment, the 3-¹⁸F-fluoropicolinamide probe includes a label, ¹⁸F, that can be used to detect, image, or otherwise identify the 3-¹⁸F-fluoropicolinamide probe, quantify the amount of 3-¹⁸F-fluoropicolinamide probe, determine the location of the 3-¹⁸F-fluoropicolinamide probe (e.g., in imaging), and combinations thereof. Fluorine-18 (t_{1/2}=109.7 min; β⁺, 99%) is an ideal short-lived PET isotope for labeling small molecules. Additional details regarding the 3-¹⁸F-fluoropicolinamide probe are described in Example 1.

### Methods of Use

Embodiments of this disclosure include, but are not limited to: methods of imaging a sample or a subject using the labeled probe (e.g., 3-¹⁸F-fluoropicolinamide probe); methods of imaging a melanin-related disease (e.g., cancer or tumor) or related biological events (e.g., hyperpigmentation and other melanin related disease), using the labeled probe (e.g., 3-¹⁸F-fluoropicolinamide probe); methods of diagnosing a melanin-related disease or related biological events using the labeled probe (e.g., 3-¹⁸F-fluoropicolinamide probe); methods of monitoring the progress of a melanin-related disease or related biological events using the labeled probe (e.g., 3-¹⁸F-fluoropicolinamide probe), and the like.

Embodiments of the present disclosure can be used to image, detect, study, monitor, evaluate, assess, and/or screen, the melanin-related melanoma or related biological events, in particular, malignant melanoma, in vivo or in vitro using the labeled probe (e.g., 3-¹⁸F-fluoropicolinamide probe).

In a particular embodiment, the 3-¹⁸F-fluoropicolinamide probe can be used in imaging melanin-related diseases (e.g., malignant melanoma). For example, the 3-¹⁸F-fluoropicolinamide probe is provided or administered to a subject in an amount effective to result in uptake of the 3-¹⁸F-fluoropicolinamide probe into the melanin-related disease or tissue of interest. The subject is then introduced to an appropriate imaging system (e.g., PET system) for a certain amount of time (e.g., this depends on radioisotope being used, for ¹⁸F, it usually could be up to 4 hours). The melanin related disease that takes up the 3-¹⁸F-fluoropicolinamide probe could be detected using the imaging system. The location of the detected signal from the 3-¹⁸F-fluoropicolinamide probe can be correlated with the location of the melanin related disease. In an embodiment, the dimensions of the location can be determined as well. Other labeled probes can be used in a similar manner.

In an embodiment, the steps of this method can be repeated at determined intervals so that the location and/or size of the disease can be monitored as a function of time and/or treatment. In particular, the 3-¹⁸F-fluoropicolinamide probe can find use in a host undergoing chemotherapy or other treatment (e.g., using a drug), to aid in visualizing the response of a disease or tumor to the treatment. In this embodiment, the 3-¹⁸F-fluoropicolinamide probe is typically visualized and sized prior to treatment, and periodically (e.g., daily, weekly, monthly, intervals in between these, and the like) during chemotherapy, radiotherapy, and the like, to monitor the tumor size. Other labeled probes can be used in a similar manner.

Embodiments of the 3-¹⁸F-fluoropicolinamide probe also find use as a screening tool in vitro to select compounds for use in treating melanin related diseased tissue or cells. The melanin related disease could be easily monitored by incubating the cells with the disease with the 3-¹⁸F-fluoropicolinamide probe during or after incubation with one or more candidate drugs. The ability of the drug compound to affect the disease can be imaged over time using the 3-¹⁸F-fluoropicolinamide probe. Other labeled probes can be used in a similar manner.

It should be noted that the amount effective to result in uptake of the labeled probe (e.g., 3-¹⁸F-fluoropicolinamide probe) into the cells or tissue of interest may depend upon a variety of factors, including for example, the age, body weight, general health, sex, and diet of the host; the time of administration; the route of administration; the rate of excretion of the specific probe employed; the duration of the treatment; the existence of other drugs used in combination or coincidental with the specific composition employed; and like factors well known in the medical arts.

### Kits

The present disclosure also provides packaged compositions or pharmaceutical compositions comprising a pharmaceutically acceptable carrier and a labeled probe (e.g., 3-¹⁸F-fluoropicolinamide probe) of the disclosure. In certain embodiments, the packaged compositions or pharmaceutical composition includes the reaction precursors to be used to generate the labeled probe according to the present disclosure. Other packaged compositions or pharmaceutical compositions provided by the present disclosure further include indicia including at least one of: instructions for using the labeled probe to image a host, or host samples (e.g., cells or tissues), which can be used as an indicator of conditions including, but not limited to, melanin related disease and biological related events.

Embodiments of this disclosure encompass kits that include, but are not limited to, the labeled probe (e.g., 3-¹⁸F-fluoropicolinamide probe) and directions (written instructions for their use). The components listed above can be tailored to the particular biological event to be monitored as described herein. The kit can further include appropriate buffers and reagents known in the art for administering various combinations of the components listed above to the host cell or host organism. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like.

### Dosage Forms

Embodiments of the present disclosure can be included in one or more of the dosage forms mentioned herein. Unit dosage forms of the pharmaceutical compositions (the “composition” includes at least the labeled probe, e.g., 3-¹⁸F-fluoropicolinamide probe) of this disclosure may be suitable for oral, mucosal (e.g., nasal, sublingual, vaginal, buccal, or rectal), parenteral (e.g., intramuscular, subcutaneous, intravenous, intra-arterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure typically vary depending on their use. For example, a parenteral dosage form may contain smaller amounts of the active ingredient than an oral dosage form used to treat the same condition or disorder. These and other ways in which specific dosage forms encompassed by this disclosure vary from one another will be readily apparent to those skilled in the art (See, e.g., Remington's Pharmaceutical Sciences, 18th ed., Mack Publishing, Easton, Pa. (1990)).

Typical compositions and dosage forms of the compositions of the disclosure can include one or more excipients. Suitable excipients are well known to those skilled in the art of pharmacy or pharmaceutics, and non-limiting examples of suitable excipients are provided herein. Whether a particular excipient is suitable for incorporation into a composition or dosage form depends on a variety of factors well known in the art including, but not limited to, the way in which the dosage form will be administered to a patient. For example, oral dosage forms, such as tablets or capsules, may contain excipients not suited for use in parenteral dosage forms. The suitability of a particular excipient may also depend on the specific active ingredients in the dosage form. For example, the decomposition of some active ingredients can be accelerated by some excipients, such as lactose, or by exposure to water. Active ingredients that include primary or secondary amines are particularly susceptible to such accelerated decomposition.

The disclosure encompasses compositions and dosage forms of the compositions of the disclosure that can include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate, or organic acids. An exemplary solubility modulator is tartaric acid.

“Pharmaceutically acceptable salt” refers to those salts that retain the biological effectiveness and properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

Embodiments of the present disclosure include pharmaceutical compositions that include the labeled probe (e.g., 3-¹⁸F-fluoropicolinamide probe), pharmaceutically acceptable salts thereof, with other chemical components, such as physiologically acceptable carriers and excipients. One purpose of a pharmaceutical composition is to facilitate administration of labeled probe (e.g., 3-18F-fluoropicolinamide probe) to a subject (e.g., human).

Embodiments of the present disclosure may be salts and these salts are within the scope of the present disclosure. Reference to a compound of any of the formulas herein is understood to include reference to salts thereof, unless otherwise indicated. The term “salt(s)”, as employed herein, denotes acidic and/or basic salts formed with inorganic and/or organic acids and bases. In addition, when an embodiment of the present disclosure contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps which may be employed during preparation. Salts of the compounds of an active compound may be formed, for example, by reacting an active compound with an amount of acid or base, such as an equivalent amount, in a medium such as one in which the salt precipitates or in an aqueous medium followed by lyophilization.

Embodiments of the present disclosure that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Embodiments of the present disclosure that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the disclosure are also contemplated herein. Solvates of the compounds are preferably hydrates.

The amounts and a specific type of active ingredient (e.g., a labeled probe such as 3-¹⁸F-fluoropicolinamide probe) in a dosage form may differ depending on various factors. It will be understood, however, that the total daily usage of the compositions of the present disclosure will be decided by the attending physician or other attending professional within the scope of sound medical judgment. The specific effective dose level for any particular host will depend upon a variety of factors, including for example, the activity of the specific composition employed; the specific composition employed; the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts. For example, it is well within the skill of the art to start doses of the composition at levels lower than those required to achieve the desired effect and to gradually increase the dosage until the desired effect is achieved.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

B16F10 was cultured in Dulbecco's modified Eagle high-glucose medium (Gibco Life Sciences) supplemented with 10% fetal bovine serum with penicillin and streptomycin. The cells were regularly maintained in a 37° C., 5% CO₂ humidified incubator. The subcutaneous B16F10 tumor models were prepared with 5- to 6-wk-old male C57BL/6 mice (Charles River). Static microPET scans were performed on the B16F10 tumor-bearing mice and representative decay-corrected coronal images at 0.5, 1, and 2 h after tail vein injection of ¹⁸F-P3BZA (3-¹⁸F-fluoropicolinamide) probe and ¹⁸F-P2BZA (2-¹⁸F-fluoronicotinamide) probe are shown in FIG. 2 (n=5 for each group), from which the B16F10 tumors were clearly visualized with good tumor-to-background contrast for both tracers (the white arrows indicate the tumors).

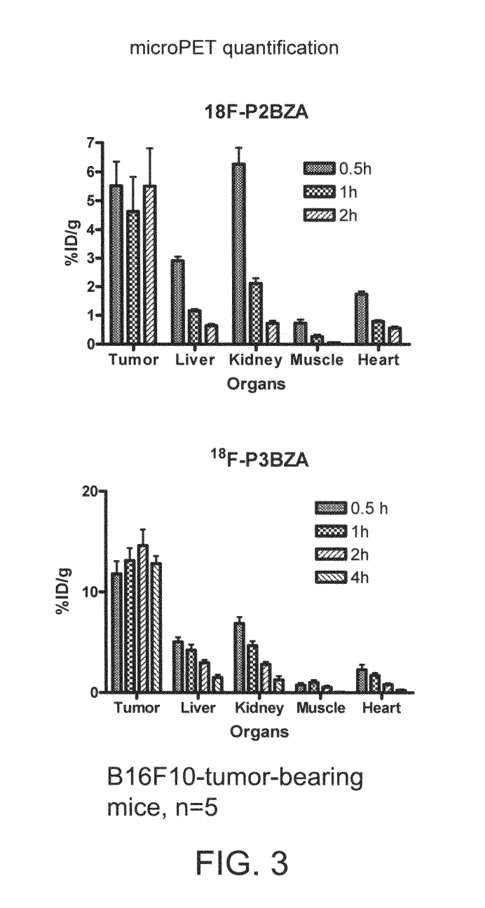

The microPET quantification analysis of tumors and other organs was obtained from the ROI analysis and the results at 0.5, 1, and 2 h p.i. are shown in FIG. 3. The tumor uptake of 18F-F3BZA probe was 11.8±2.2, 13.2±2.4, and 14.5±3.2% ID/g at 0.5, 1, and 2 h p.i., respectively. For ¹⁸F-P2BZA probe, the tumor uptake was 5.5±1.6, 4.6±2.4, 5.5±2.6% ID/g at 0.5, 1, and 2 h p.i., respectively. At each time point the tumor uptakes of ¹⁸F-P3BZA probe was significantly higher than those of ¹⁸F-P2BZA probe (P<0.05). While for the previous ¹⁸F-F3ZA probe data, the tumor uptakes are only 6.5±2.2, and 5.9±1.8% ID/g at 1 h and 2 h post injection, which are significantly lower than the corresponding time point of ¹⁸F—P3BZA. ¹⁸F-P3BZA probe was cleared rapidly from the blood, and moderate to low kidney and liver uptakes were observed (Kidneys: 6.9±1.1, 4.7±0.8, 3.0±0.6% ID/g at 0.5, 1, and 2 h p.i. Liver: 5.1±0.8, 4.3±1.1, 3.0±0.6% ID/g at 0.5, 1, and 2 h p.i.). The unspecific uptake by muscle is very low, 0.8±0.4, 1.0±0.5, 0.6±0.3% ID/g at 0.5, 1, and 2 h p.i.), which is the explanation of the high contrast images shown in FIG. 2.

### Example 2

In this example ¹⁸F-picolinamide probes (¹⁸F-P3BZA and ¹⁸F-P4BZA probes) were synthesized and evaluated for the imaging of malignant melanoma.

The ¹⁸F-fluoropicolinamides were prepared by radiofluorination reactions using no-carrier-added ¹⁸F-fluoride and evaluated in vivo by biodistribution studies and micro-positron emission tomography (microPET) imaging in C57BL/6 mice bearing B16F10 murine melanoma tumors.

¹⁸F-fluorination of the corresponding bromopicolinamide precursors was achieved in yields of 10-20% (decay corrected on the basis of ¹⁸F-fluoride). The total synthesis time was less than 1 hour. The radiochemical purity of the ¹⁸F-labeled probes was more than 98% and the specific activity was 100-150 GBq/μmol. Noninvasive microPET and direct tissue sampling experiments demonstrated that the probes had melanin-specific tumor targeting in subcutaneous B16F10 melanoma xenografts, which yielded a tumor-to-blood ratio of approximately 20:1 at 1 h and greater than 30:1 at 2 h. Quantitative microPET imaging studies showed that ¹⁸F-P3BZA had tumor uptake of 11.8±2.2, 13.2±2.4, and 14.6±3.2% ID/g at 0.5 h, 1 h, and 2 h postinjection, respectively. Of note, the ¹⁸F-P3BZA demonstrated good in vivo stability as evidenced by the low bone uptake in biodistribution studies.

The ¹⁸F-picolinamide probes were synthesized with high specific activity via one-step ¹⁸F-fluorination within a short time frame. The probes exhibited high tumor uptake and metabolic stability, as well as favorable in vivo pharmacokinetics. These findings suggest that ¹⁸F-fluoropicolinamides could be used as imaging agents for the detection of melanoma

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are merely set forth for a clear understanding of the principles of this disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

We claim at least the following:

1. A method of diagnosing the presence of a melanin related disease in a subject comprising:
administering to the subject a labeled probe;
imaging at least a portion of the subject; and
detecting the labeled probe, wherein the location of the labeled probe corresponds to a melanin related disease,
wherein the labeled probe has the following structure: 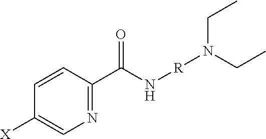
wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, or ¹⁵³Sm, and R is selected from the group consisting of: a substituted or unsubstituted, linear or branched, CₙH₂ₙ, where n is 1 to 12, and a substituted or unsubstituted, linear or branched, alkene group having 2 to 12 carbons.

2. The method of claim 1, wherein the melanin related disease is melanoma.

3. The method of claim 1, wherein the labeled probe is a 3-¹⁸F-fluoropicolinamide probe.

4. A method of diagnosing the presence of a melanin related disease in a sample comprising:
contacting the sample with a labeled probe;
imaging at least a portion of the sample; and
detecting the labeled probe, wherein the location of the labeled probe corresponds to a melanin related disease,
wherein the labeled probe has the following structure: 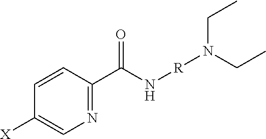
wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, or ¹⁵³Sm, and R is selected from the group consisting of: a substituted or unsubstituted, linear or branched, CₙH₂ₙ, where n is 1 to 12, and a substituted or unsubstituted, linear or branched, alkene group having 2 to 12 carbons.

5. The method of claim 4, wherein the sample is selected from a tissue or a cell.

6. The method of claim 4, wherein the labeled probe is a 3-¹⁸F-fluoropicolinamide probe.

7. A method of monitoring the progress of a melanin related disease in a subject comprising:
administering to the subject a labeled probe;
imaging at least a portion of the subject; and
detecting the labeled probe, wherein the location of the labeled probe corresponds to a melanin related disease, wherein the dimensions of the location is monitored over time,
wherein the labeled probe has the following structure: 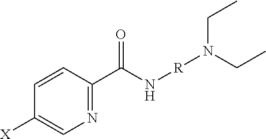
wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, or ¹⁵³Sm, and R is selected from the group consisting of: a substituted or unsubstituted, linear or branched, CₙH₂ₙ, where n is 1 to 12, and a substituted or unsubstituted, linear or branched, alkene group having 2 to 12 carbons.

8. The method of claim 7, further comprising repeating the steps of claim 4 periodically to monitor the dimensions of the location corresponding to the melanin related disease.

9. The method of claim 7, wherein the labeled probe is a 3-¹⁸F-fluoropicolinamide probe.

10. The method of claim 7, wherein the melanin related disease is melanoma.

11. A method of monitoring the progress of a melanin related disease in a sample comprising:
contacting the sample with a labeled probe;
imaging at least a portion of the sample; and
detecting the labeled probe, wherein the location of the labeled probe corresponds to a melanin related disease, wherein the size of the location is monitored over time,
wherein the labeled probe has the following structure: 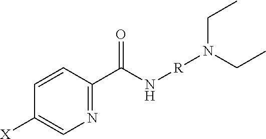
wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, or ¹⁵³Sm, and R is selected from the group consisting of: a substituted or unsubstituted, linear or branched, CₙH₂ₙ, where n is 1 to 12, and a substituted or unsubstituted, linear or branched, alkene group having 2 to 12 carbons.

12. The method of claim 11, wherein the sample is selected from a tissue or a cell.

13. The method of claim 11, wherein the labeled probe is a 3-¹⁸F-fluoropicolinamide probe.

14. A method of screening for an agent for treating a melanin related disease in a sample comprising:
contacting the sample with a labeled probe, wherein a melanin related disease is present in the sample;
contacting an agent with the sample;
imaging at least a portion of the sample; and
detecting the labeled probe, wherein the location of the labeled probe corresponds to a melanin related disease, wherein the size of the location is monitored over time,
wherein the labeled probe has the following structure: 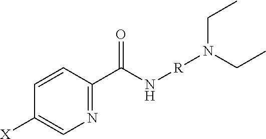
wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, or ¹⁵³Sm, and R is selected from the group consisting of: a substituted or unsubstituted, linear or branched, CₙH₂ₙ, where n is 1 to 12, and a substituted or unsubstituted, linear or branched, alkene group having 2 to 12 carbons.

15. The method of claim 14, wherein the sample is selected from a tissue or a cell.

16. The method of claim 14, wherein the labeled probe is a 3-¹⁸F-fluoropicolinamide probe.

17. A probe, comprising: a labeled probe, wherein the labeled probe has the following structure: 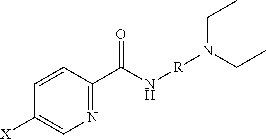
wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, or ¹⁵³Sm, and R is selected from the group consisting of: a substituted or unsubstituted, linear or branched, CₙH₂ₙ, where n is 1 to 12, and a substituted or unsubstituted, linear or branched, alkene group having 2 to 12 carbons.

18. The probe of claim 17, wherein the labeled probe is a 3-¹⁸F-fluoropicolinamide probe.

19. A composition, comprising:
a labeled probe, wherein the labeled probe has the following structure: 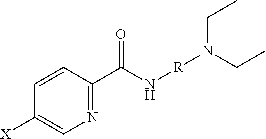
wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, or ¹⁵³Sm, and R is selected from the group consisting of: a substituted or unsubstituted, linear or branched, CₙH₂ₙ, where n is 1 to 12, and a substituted or unsubstituted, linear or branched, alkene group having 2 to 12 carbons.

20. The composition of claim 19, wherein the labeled probe is a 3-¹⁸F-fluoropicolinamide probe.

21. A pharmaceutical composition, comprising:
a pharmaceutical carrier and an effective dose of a labeled probe, wherein the labeled probe has the following structure: 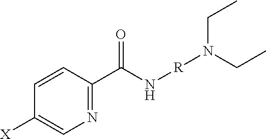
wherein X is selected from the group consisting of: ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, or ¹⁵³Sm, and R is selected from the group consisting of: a substituted or unsubstituted, linear or branched, CₙH₂ₙ, where n is 1 to 12, and a substituted or unsubstituted, linear or branched, alkene group having 2 to 12 carbons.

22. The pharmaceutical composition of claim 21, wherein the labeled probe is a 3-¹⁸F-fluoropicolinamide probe.
